(19) United States
(12) Patent Application Publication
(10) Pub. No.: US 2007/0065438 A1
(43) Pub. Date: Mar. 22, 2007

(54) Methods of inducing and maintaining immune tolerance

(75) Inventors: Janet Liversidge, Aberdeenshire (GB); Andrew Dick, Bristol (GB); Jonathon Sedgwick, Indianapolis, IN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/599,798

(22) Filed: Nov. 15, 2006

Related U.S. Application Data
(63) Continuation of application No. 10/741,430, filed on Dec. 18, 2003, now abandoned.
(60) Provisional application No. 60/436,739, filed on Dec. 27, 2002.

(51) Int. Cl. A61K 48/00; A61K 39/395

(52) U.S. Cl. 424/144.1; 514/44

(57) ABSTRACT

Provided are methods of enhancing and maintaining immune tolerance by modulation of CD200 or CD200R. Provided are antagonists thereof including antibodies.

This application claims benefit of U.S. Provisional patent application Ser. No. 60/436,739, filed Dec. 27, 2002.

### FIELD OF THE INVENTION

The present invention relates to methods for modulating mammalian physiology, including the immune system function. In particular, it provides methods for inducing and maintaining immune tolerance using modulators of CD200 or CD200R.

### BACKGROUND OF THE INVENTION

Regulation of immune response to infection or injury involves initiation signals, as well as termination signals that restore and maintain immunological homeostasis. These regulatory processes can involve gene families that encode related receptors with opposing functions that allow fine-tuning of the immune response to antigen challenge. Antigen presenting cells (APC) of the myeloid lineage, such as macrophages and dendritic cells (DC), are central to these regulatory processes. The importance of this modulation is demonstrated by the sometimes fatal autoimmune and lymphoproliferative disorders observed in mice with targeted disruption of inhibitory receptors. The ligands B7.1 and B7.2 for CD28 and CTLA-4 represent a vital control point for T cells and in myeloid cells the signal regulating proteins SIRPα and SIRPβ have been identified with reciprocal roles in myeloid cell function. CD200, also known as OX-2, has been identified as a possible negative regulator of both myeloid antigen presenting cells and activated T lymphocytes. Alternatively, CD200 which has sequence homology to B7.1 and B7.2 molecules, is reported to function as a co-stimulatory molecule inducing T cell proliferation, but with altered cytokine secretion patterns. Thus, similar to other recently described negative co-receptors, CD200 may exert different effects at different points in the immune response, although the mechanisms involved are unknown at present (see, e.g., Lanier (2001) Curr. Opin. Immunol. 13:326-331; Goerdt and Orfanos (1999) Immunity 10: 137-142; Ravetch and Lanier (2000) Science 290:84-89; Tivol, et al. (1996) Curr. Opin. Immunol. 8:822-830; Cant and Ullrich (2001) Cell Mol. Life Sci. 58:117-124; Dietrich, et al. (2000) J. Immunol 164:9-12; Barclay and Ward. (1982) Eur. J. Biochem. 129:447458; Hoek, et al. (2000) Science 290:1768-1771; Wright, et al. (2001) Immunology 102:173-179; Gorczynski, et al. (2000) J Immunol. 165:48544860; Borriello, et al. (1997) J. Immunol. 158:45484554; and Borriello, et al. (1998) Mamm. Genome 9:114-118; Greenwald, et al. (2002) Curr. Opin. Immunol. 14:391-396).

CD200 is a widely distributed membrane-bound protein occurring on lymphoid, including re-circulating B cells and activated but not resting T cells, neuronal, endothelial, and dendritic cells. Human CD200 is expressed similarly, including in normal brain and by B cells. This membrane bound ligand is distinguished by its short cytoplasmic domain (19 amino acids). CD200 of one cell can bind to CD200 receptor (CD200R; OX2R) of a separate cell. In humans, two subtypes of CD200Rs have been identified, hCD200Ra and hCD200Rb while the mouse homolog consists of four receptor subtypes, CD200Ra, CD200Rb, CD200Rc, and CD200Rd. CD200Ra is expressed predominantly on macrophages, microglia (macrophages of brain), monocytes, and granulocytes (see, e.g., Wright, et al., supra; Hoek, et al. (2000) Science 290:1768-1771; McCaughan, et al. (1987) Immunogenetics 25:329-335).

CD200 deficient mice (a.k.a. CD200^{-/-}; CD200 knockout; CD200KO) exhibit various myeloid defects. These defects include elevated numbers of macrophages within tissues normally expressing CD200, and increased DAP-12 expression particularly in the marginal zone of secondary lymphoid tissues, indicating myeloid cell activation. As a consequence of this phenotype, mice lacking CD200 appear to have increased susceptibility CD4⁺ T cell mediated autoimmune diseases. In particular, CD200/CD200R regulation of microglial activation has profound effects on neuronal tissues, accelerating onset of experimental models of autoimmunity affecting the central nervous system including experimental autoimmune encephalomyelitis (EAE) and experimental autoimmune uveoretinitis (EAU) (see, e.g., Hoek, et al., supra; Broderick, et al. (2002) Am. J. Pathol. 161:1669-1677).

EAU is mediated by retinal antigen specific CD4⁺ T cells and can be modulated using various therapeutic approaches targeting T helper cell function including induction of antigen specific tolerance via the nasal mucosa. Activated macrophages are required for full expression of disease, but equally, macrophages are required for resolution of inflammation. For instance, macrophages respond to signals such as IL-4 and IL-10 and actively participate in the anti-inflammatory process supporting the concept of the alternatively activated macrophage having a role in healing and tissue remodelling. Such alternatively activated macrophages have recently been described by us in the rat model of EAU. Myeloid APC may also have a dual role in nasal tolerance induction in EAE and EAU where signalling by neuronally expressed CD200 must occur during the inflammatory process. In these models effective protection is associated with an initial IFNgamma driven priming event in cervical lymph nodes followed by T cell apoptosis and a down regulation of the capacity of antigen specific T cells to proliferate in response to re-stimulation (see, e.g., Dick (2000) Int. Ophthalmol. Clin. 40:1-18; Dick (1999) Dev. Ophthalmol. 30:187-202; Dick, et al. (1994) Immunology 82:625-631; Dick, et al. (2001) Br. J. Ophthalmol. 85:1001-1006; Jiang, et al. (2001) Br. J. Ophthalmol. 85:739-744; Burkhart, et al. (1999) Int. Immunol. 11:1625-1634; Laliotou, et al. (1999) J. Autoimmun. 12:145-155; Jiang, et al. (1999) Invest Ophthalmol. Vis. Sci. 40:3177-3185; Dick, et al. (1996) Eur. J. Immunol. 26:1018-1025; and Liversidge, et al. (2002) Am. J. Path. 160:1-12; Stein, et al. (1992) J. Exp. Med. 176:287-292; Stumpo, et al. (1999) Pathobiology 67:245-248; and Erwig, et al. (1998) J. Immunol. 161:1983-1988).

The mechanisms underlying the induction and maintenance of tolerance are poorly understood. The present invention provides methods for inducing and maintaining tolerance through the modulation of CD200 or CD200R.

### SUMMARY OF THE INVENTION

The present invention is based, in part, upon the discovery that tolerance to an antigen can be increased by an antagonist to CD200.

The invention provides a method of modulating tolerance to an antigen in a subject with an inflammatory or immune condition or disorder, comprising treating with an agonist or antagonist of CD200. Also provided is the above method, wherein the modulating increases or maintains tolerance and the treatment comprises administering an antagonist of CD200; or increases TH2-type response; as well as the above method wherein the agonist or antagonist is derived from the antigen binding site of an antibody that specifically binds to CD200 or to CD200R; and the above method wherein the antagonist is an antibody that specifically binds to CD200; or CD200R.

In another aspect, the invention provides the above method wherein the agonist or antagonist comprises a polyclonal antibody; a monoclonal antibody; a humanized antibody; an Fv, Fab, or F(ab′)₂ fragment; a blocking antibody; or a peptide mimetic of an antibody; as well as the above method wherein the agonist or antagonist comprises a nucleic acid that encodes a CD200 or CD200R; or specifically binds a polynucleotide encoding a CD200 or CD200R; and the above method wherein the nucleic acid comprises an anti-sense nucleic acid; comprises an RNA interference nucleic acid; or genetic mutation in the genome of the subject that reduces expression of biologically active CD200 or CD200R.

Another embodiment of the invention provides a method of modulating tolerance to an antigen in a subject with an inflammatory or immune condition or disorder, comprising treating with an agonist or antagonist of CD200; wherein the condition or disorder comprises an autoimmune condition or disorder; the above method wherein the condition or disorder comprises uveoretinitis; graft or transplant rejection; diabetes mellitus; multiple sclerosis; inflammatory bowel disorder (IBD); rheumatoid arthritis; or asthma or allergy; as well as the above method wherein tolerance is induced intranasally; enterally; orally; parenterally; intravenously; or mucosally.

Still another embodiment of the present invention the provides the above method wherein the increase or maintenance comprises an improvement in a histological score; and the above method wherein the improvement comprises a reduction in inflammatory cell infiltrate; or a reduction in structural tissue damage; as well as the above method wherein the cell infiltrate is in a retina; or the tissue damage is of a photoreceptor cell; and the above method wherein the disorder or condition results from an immunization.

Yet another aspect of the present invention provides the above method wherein the TH2-type response comprises a detectable increase in expression or levels of a cytokine that is IL-4; IL-5; IL-10; or IL-13; as well as the above method wherein expression or levels of the TH2 cytokine is at least 2-fold greater with CD200 antagonist treatment than without CD200 antagonist treatment; and the above method wherein the condition or disorder results from an immunization and where the at least 2-fold greater expression or levels occurs on or before day 21 after immunization.

Moreover, provided is the above invention wherein immune cell proliferation is detectably decreased or inhibited in a tolerized subject treated with a CD200 antagonist, relative to a tolerized subject not treated with a CD200 antagonist; as well as the above invention wherein immune cell proliferation with CD200 antagonist treatment is 75% or less; or 50% or less, than proliferation without CD200 antagonist treatment and; in addition; the above method wherein the immune cell is a splenocyte.

In another embodiment, the invention embraces the above method wherein the CD200 antagonist treatment results in a detectable increase in expression of STAT6; or activation of STAT6, with treatment with the CD200 antagonist, as compared with treatment without the CD200 antagonist, the above method wherein there is a detectable increase in activity or levels of T regulatory cells (Tregs); or IL-10-expressing cells; with treatment with the CD200 antagonist, as compared with treatment without the CD200 antagonist; as well as the above method wherein the Tregs comprise CD3⁺CD4⁺CD25⁺T cells; or the IL-10 expressing cells are CD11b⁻; CD11b⁻, CD11c^{-/low}, CD3⁻, B220⁻, CD45RB^{intermediate}; or plasmacytoid dendritic cells.

The invention provides a method of modulating tolerance to an antigen in a subject with an inflammatory or immune condition or disorder, comprising treating with an agonist or antagonist of CD200; wherein the modulating is decreasing and the treating comprises an agonist of CD200; the above method wherein the immune condition or disorder is persistent infection; or cancer; as well as the above method wherein the modulation decreases TH2 response; or decreases or inhibits activity or levels of regulatory T cells (Tregs).

### DETAILED DESCRIPTION

As used herein, including the appended claims, the singular forms of words such as “a,” “an,” and “the” include their corresponding plural references unless the context clearly dictates otherwise.

All references cited herein are incorporated herein by reference to the same extent as if each individual publication, patent, or published patent application was specifically and individually indicated to be incorporated by reference.

I. Definitions.

An “antagonist” or “inhibitor,” or “agonist” or “activator,” refers to inhibitory or activating molecules, respectively, as it pertains to the modulation of activity of, e.g., a ligand, receptor, cofactor, a gene, cell, tissue, or organ. A modulator of, e.g., a gene, receptor, ligand, or cell, is a molecule that alters an activity of the gene, receptor, ligand, or cell, where activity can be activated, inhibited, or altered. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to a control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher than the control.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 80% of the control or less, more generally 70% of the control or less, most generally 60% of the control or less, preferably 50% of the control or less, more preferably 40% of the control or less, and most preferably 30% of the control or less, usually 20% of the control or less, more usually 10% of the control or less, and most usually 5% of the control or less. Generally, an endpoint of activation is at least 150% the control, preferably at least two times the control, more preferably at least four times the control, and most preferably at least 10 times the control.

As used herein, the term “biological activity” is used to describe, without limitation, metabolic, signaling, hormonal, developmental, embryological, proliferative, apoptotic, secretory, migratory, adhesive, neurological, pathological, inflammatory, and cancerous activities of a cell, tissue, organ, or animal, a cultured cell or tissue, a perfused tissue or organ, or animal sustained on life support. “Biological activity” also includes the catalytic activity of enzymes in vivo and enzymes in the purified state, as well as changes in conformation in enzymes and other proteins.

“Biological compartment” refers to a tissue, organ, cell, organelle, or component of a cell, for example, a lymph node, an endothelial or epithelial layer, or a region of the spleen, e.g., red pulp or white pulp. “Biological compartment” also can refer to the fluid, colloid, gel, or slurry contained within or derived from a given compartment, such as cytosol, nucleosol, cerebrospinal fluid, plasma, serum, whole blood, urine, bile, or lymph.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist irradication by the immune system. “Proliferative condition” encompasses, e.g., cancer, cancer cells, tumors, angiogenesis, precancerous conditions such as dysplasia, as well as conditions by proliferation, e.g., of bacteria, parasites, multicellular foreign organisms, and viruses.

“Specifically” or “selectively” binds, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two fold greater, preferably at least ten times greater, more preferably at least 20-times greater, and most preferably at least 100-times greater than the affinity with any other antibody, or binding composition derived thereof. In a preferred embodiment the antibody will have an affinity that is greater than about 10⁹ liters/mol, as determined, e.g., by Scatchard analysis (Munsen, et al. (1980) Analyt. Biochen. 107:220-239).

“Splenocytes” are cells harvested from the spleen that comprise T cells, B cells, monocytes, NK cells, and/or others (see, e.g., Metwali, et al. (2002) Am. J. Physiol. Gastrointest. Liver Physiol. 283:G115-G121; Schaefer, et al. (2001) J. Immunol. 166:5859-5863; Hameg, et al. (1999) J Immunol. 162:7067-7074). In studies of splenocyte proliferation, generally the T cell is the cell most active in proliferation. The invention contemplates a method of treating with a CD200 antagonist, wherein proliferation of splenocytes, T cells, immune cells, or immune cells derived from the bloodstream, e.g., PBMCs, is decreased generally by 10% or more, more generally by 20% or more, most generally by 30% or more, typically by 40% or more, more typically by 50% or more, most typically by 60% or more, usually by 70% or more, more usually by 80% or more, and most usually by 90% or more.

“Ligand” refers to a small molecule, peptide, polypeptide, or membrane associated and membrane-bound molecule that act as an agonist, antagonist, or binding agent of a receptor. Ligand also encompasses soluble versions of said membrane-associated ligand or membrane-bound ligand. Where the ligand is membrane-bound on a first cell, the receptor usually occurs on a second cell. The second cell may have the same or a different identity as the first cell. Ligands and receptors may be entirely intracellular, that is, it may reside in the cytosol, nucleus, or some other intracellular compartment. The complex of a ligand and receptor is termed a “ligand receptor complex.” Where a ligand and receptor are involved in a signaling pathway, the ligand occurs at an upstream position and the receptor occurs at a downstream position of the signaling pathway. Methods for determining ligand to receptor binding constants and kinetic properties are available (Karlsson, et al. (1991) J. Immunol. Methods 145:229-240; Neri, et al. (1997) Nature Biotechnology 15:1271-1275; Jonsson, et al. (1991) Biotechniques 11:620-627; Friguet, et al. (1985) J. Immunol. Methods 77:305-319; Hubble (1997) Immunol. Today 18:305-306).

“Nucleic acid” encompasses single stranded polynucleotides, e.g., ssDNA, double stranded polynucleotides, e.g., dsDNA, and multistranded polynucleotides, as well as probes and primers. The invention also provides modified nucleic acids, e.g., biotinylated nucleic acids, molecular beacons, anti-sense nucleic acids, compositions for RNA interference, and peptide-nucleic acids (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189).

“Therapeutically effective amount” of a therapeutic agent is defined as an amount of each active component of the pharmaceutical formulation that is sufficient to show a meaningful patient benefit, i.e., to cause a decrease in or amelioration of the symptoms of the condition being treated. When the pharmaceutical formulation comprises a diagnostic agent, “a therapeutically effective amount” is defined as an amount of each active component of the pharmaceutical formulation that is sufficient to produce an image or other diagnostic parameter in the diagnostic system employed. When applied to an individual active ingredient, administered alone, the term refers to that ingredient alone. When applied to a combination of active ingredients, the term refers to combined amounts of the active ingredients that result in the therapeutic effect, whether administered in combination, serially or simultaneously. Effective amounts of the pharmaceutical formulation will vary according to factors such as the degree of susceptibility of the individual, the age, sex, and weight of the individual, and idiosyncratic responses of the individual (see, e.g., U.S. Pat. No. 5,888,530).

“Tolerance” encompasses immune unresponsiveness to, e.g., a cancer or tumor, or to an alloantigen, such as a graft alloantigen, to a foreign antigenic molecule, to a foreign molecular complex, or to an antigen from a foreign organism or virus. Tolerance also encompasses immune unresponsiveness, or an increase in immune unresponsiveness, to an autoantigen, e.g., where the autoantigen pertains to an autoimmune disorder. Additionally, “tolerance” encompasses naturally occurring tolerance and artificially or pharmacologically induced tolerance. Moreover, tolerance also relates to immune unresponsiveness to self-antigens that are recognized by molecular mimicry (see, e.g., Liu (1997) J. Exp. Med. 186:625-629; Waldman and Cobbold (1998) Annu. Rev. Immunol. 16:619-644; Xiao and Link (1997) Clin. Immunol. Immunopathol. 85:119-128; Steinman, et al. (2003) Annu. Rev. Immunol. 21:685-711; Olson, et al. (2002) J. Immunol. 169:2719-2726; Toussirot (2002) Curr. Drugs Targets Inflamm. Allergy 1:45-52; Takahashi and Sakaguchi (2003) Int Rev Cytol. 225:1-32; Burt, et al. (2002) Int J Hematol. 76 (Suppl 1):226-47; Gery and Egwuagu (2002) Int. Rev. Immunol. 21(2-3):89-100; Weiner (2001) Microbes Infect. 3:947-54).

Reduction of tolerance, e.g., by administering an agonist of CD200, that is, an agonist of the CD200/CD200R signaling pathway, is useful, e.g., for reducing TH2 response, e.g., in the treatment of persistent infections, such as malaria, or for the treatment of tumors, cancers, neoplasms, and viruses, including persistant tumors, cancers, neoplasms, and viruses. Persistant candidiasis infections are associated with TH2 response (see, e.g., Lilic, et al. (1996) Clin. Exp. Immunol. 105:205-212; Engelhard, et al. (2002) Immunol. Rev. 188:136-146; Good (1995) Parasite Immunol. 17:55-59; Liu, et al. (2002) Mol. Cancer Ther. 1:1147-1151; Sakaguchi, et al. (2001) Immunol. Rev. 182:18-32; Kakimi, et al. (2002) J. Virol. 76:8609-8620).

The invention is not limited by the mechanism by which tolerance is mediated. Encompassed are methods of modulating tolerance, e.g., by modulating an activity or property of regulatory T cells (Tregs), such as CD4⁺CD25⁺ T cells, Tr1 cells; Th3 cells, CD8⁺ suppressor T cells, or gamma delta T cells; by antigen presenting cells (APCs), such as dendritic cells; or by T cell anergy (see, e.g., Ohashi and DeFranco (2002) Curr. Opinion Immunol. 14:744-759; Kuwana (2002) Hum. Immunol. 63:1156-1163; Gilliet and Liu (2002) Hum. Immunol. 63:1149-1155; Turley (2002) Curr. Opin. Immunol. 14:765-770; Ke, et al. (1997) J. Immunol. 58:3610-3618).

The invention contemplates modulation of tolerance by modulating TH1 response, TH2 response, or both TH1 and TH2 response. Modulating TH1 response encompasses changing expression of, e.g., interferon-gamma. Modulating TH2 response encompasses changing expression of, e.g., any combination of IL-4, IL-5, IL-10, and IL-13. Typically an increase (decrease) in TH2 response will comprise an increase (decrease) in expression of at least one of IL-4, IL-5, IL-10, or IL-13; more typically an increase (decrease) in TH2 response will comprise an increase in expression of at least two of IL-4, IL-5, IL-10, or IL-13, most typically an increase (decrease) in TH2 response will comprise an increase in at least three of IL-4, IL-5, IL-10, or IL-13, while ideally an increase (decrease) in TH2 response will comprise an increase (decrease) in expression of all of IL-4, IL-5, IL-10, and IL-13.

Also contemplated is modulation of “infectious tolerance,” where transfer of T cells from one subject to another transfers tolerance (see, e.g., Unger, et al. (2003) Int. Immunol. 15:731-739; Iwashiro, et al. (2001) Proc. Natl. Acad. Sci. USA 98:9226-9230).

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a CD200 or CD200R agonist or antagonist to a human or animal subject, a cell, tissue, physiological compartment, or physiological fluid. “Treatment of a cell” also encompasses situations where the CD200 or CD200R agonist or antagonist contacts CD200 or CD200R, e.g., in the fluid phase or colloidal phase, but also situations where the agonist or antagonist administered has not been demonstrated to contact the cell, the CD200, or the CD200R.

II. General.

Inhibition of immune response, as occurs in tolerance, is mediated by myeloid cells, e.g., DCs and macrophages, and lymphoid cells, e.g., regulatory T cells (Tregs) such as CD4⁺CD25⁺ T cells and Tr1 cells. Alternative macrophage activation and scavenger receptor expression are among the mechanisms of immune response inhibition. Cytokines such as IL-10 can modulate inhibition of immune response, and the importance of this response is demonstrated by the sometimes fatal autoimmune and lymphoproliferative disorders observed in mice with targeted disruption of inhibitory receptors or IL-10 signaling (see, e.g., Moore, et al. (2001) Annu. Rev. Immunol. 19:683-765; McGuirk, et al. (2002) J. Exp. Med. 195:221-231; Kaya, et al. (2002) J. Immunol. 168:1552-1556; Lanier (2001) Curr. Opin. Immunol. 13:326-331; Colonna (2003) Nat. Rev. Immunol. 3:445453; Goerdt and Orfanos (1999) Immunity 10:137-142; Kuhn, et al. (1993) Cell 75:263-274).

CD200, a negative regulator of immune function, is expressed by a variety of cells including neurons, microvascular endothelium, re-circulating B cells and activated but not resting T cells, while its structurally related inhibitory receptor (CD200R) is restricted to cells of the myeloid lineage including monocyte/macrophages, DC and microglia and some T lymphocytes. Two additional members of the CD200R family, mCD200RLa and mCD200Lb, occur in mice. mCD200RLa and mCD200Lb do not bind to CD200 but have a potential activating function through DAP-12 adapter protein binding. Thus, in common with other recently described negative co-receptors, CD200 may exert different effects at different points in the immune response (see, e.g., Cant and Ullrich (2001) Cell Mol. Life. Sci. 58:117-124; Dietrich, et al. (2000) J. Immunol. 164:9-12; Barclay and Ward (1982) Eur. J. Biochem. 129:447-458; Wright, et al. (2001) Immunology 102:173-179; Hoek, et al. (2000) Science 290:1768-1771; Gorczynski, et al. (2000) J. Immunol. 165:48544860; Gorczynski, et al. (2000) Clin. Immunol. 97:69-78; Preston, et al. (1997) Eur. J. Immunol. 27:1911-1918; Wright, et al. (2000) Immunity 13:233-242; Dick, et al. (2001) Invest. Ophthalmol. Vis. Sci. 42:170-176; Wright, et al. (2003) J. Immunol. 171:3034-3046; Greenwald, et al. (2002) Curr. Opin. Immunol. 14:391-396).

CD200KO mice exhibit various myeloid defects, e.g., elevated numbers of macrophages within tissues normally expressing CD200, and increased DAP-12 expression particularly in the marginal zone of secondary lymphoid tissues, indicating myeloid cell activation. CD200KO mice appear to have increased susceptibility to CD4⁺ T cell mediated autoimmune diseases. In particular, CD200 and CD200R-mediated regulation of microglial activation has marked effects on neuronal tissues, accelerating onset of experimental models of autoimmunity affecting the central nervous system, e.g., experimental autoimmune encephalomyelitis (EAE) and experimental autoimmune uveoretinitis (EAU) (see, e.g., Broderick, et al. (2002) Am. J. Pathol. 161:1669-1677).

EAU is mediated by retinal antigen specific CD4⁺ T cells, where EAU can be modulated using therapeutic approaches targeting T helper cell function, e.g., induction of antigen specific tolerance via the nasal mucosa. Activated macrophages are required for full expression of disease, but equally, macrophages are required for resolution of inflammation. In the resolution of inflammation, macrophages respond to signals such as IL-4 and IL-10. Myeloid APC may also have a dual role in nasal tolerance induction in EAE and EAU. In these models protection is associated with an initial IFNgamma driven priming event in cervical lymph nodes followed by T cell apoptosis and a down regulation of the ability of antigen specific T cells to proliferate in response to re-stimulation.

The study of the present invention uses a model of tolerance, where tolerance is induced by respiratory exposure to antigen, and where CD200/CD200R-mediated signaling is shown to modulate inflammation and tolerance. The moderately susceptible C57B1/6 mouse EAU model was used because uveitogenic T cells alone are insufficient to cause target organ damage. Monocyte macrophages are also necessary and prominent in the earliest inflammatory infiltrates in the retina and, in addition, monocyte expression of NOS2 is required for full expression of disease. Respiratory tract dendritic cells (RTDC) and alveolar macrophages mediate tolerance induced by respiratory exposure to antigen.

In the study of the present invention, T cell activation and proliferation in the draining cervical lymph were followed by systemic generation of regulatory cells in the spleen (see, e.g., Dick, et al. (2000) Int. Ophthalmol. Clin. 40:1-18; Dick, et al. (1999) Dev. Ophthalmol. 30:187-202; Dick, et al. (2001) Br. J. Ophthalmol. 85:1001-1006; Jiang, et al. (2001) Br. J. Ophthalmol. 85:739-744; Jiang, et al. (1999) Invest. Ophthalmol. Vis. Sci. 40:3177-3185; Dick (1996) Eur. J. Immunol 26:1018-1025; Liversidge, et al. (2002) Am. J. Path. 160:1-12; Stein, et al. (1992) J. Exp. Med. 176:287-292; Stumpo, et al. (1999) Pathobiology 67:245-248; Erwig, et al. (1998) J. Immunol 161:1983-1988; Robertson, et al. (2002) Invest. Ophthalmol. Vis. Sci. 43:2250-2257; Burkhart, et al. (1999) Int. Immunol. 11: 1625-1634; Laliotou, et al. (1999) J. Autoimmum. 12:145-155; Avichezer, et al. (2000)

Invest Ophthalmol. Vis. Sci. 41:127-131; Forrester, et al. (1998) Curr. Eye Res. 17:426437; Dick, et al. (1996) Eur. J. Immunol. 26:1018-1025; Hoey, et al. (1997) J. Immunol 159:5132-5142; Akbari, et al. (2001) Nat. Immunol. 2:725-731; Prakken, et al. (2002) Arthritis Rheum. 46:1937-1946; Dick, et al. (1994) Eye 8 (Pt 1):52-59; Massey, et al. (2002) Vet. Immunol. Immunopathol. 87:357-372; Akbari, et al. (2001) Nat. Immunol. 2:725-731; Stumbles, et al. (1998) J. Exp. Med. 188:2019-2031).

In the study of the present invention, despite accelerated disease onset, overall disease incidence and severity was reduced over time in CD200KO mice, where reduction in disease symptoms correlated with elevated numbers of regulatory T cells and the presence of high IL-10 secreting splenic myeloid cells later in the disease process. The CD200KO enhanced tolerance to retinal antigen. This result of the CD200KO may be related to the altered phenotype of APC in the respiratory tract compared to wild type and an enhanced Th2 switch in tolerised CD200KO mice. Tolerance induction in the CD200KO mouse was efficient, with up 50% of eyes still protected from disease 28 days post-immunisation (see, e.g., Murphy and Reiner (2002) Nat. Rev. Immunol. 2:933-944; Suri-Payer, et al. (1998) J. Immunol. 160:1212-1218; Thornton and Shevach (2000) J. Immunol. 164:183-190; Roncarolo, et al. (2001) Immunol. Rev. 182:68-79; Peiser and Gordon (2001) Microbes Infect. 3:149-159; Gordon (2003) Nat. Rev. Immunol. 3:23-35).

In the studies of the present invention, there was a clear increase in CD11b⁻IL10^{high} cells in the spleens of both sham tolerised and tolerised CD200KO mice at day 28. These cells were distinct from larger populations of CD11b⁺IL10^{low} present in all experimental groups from day 21. The high level of IL-10 detected was endogenous as cells were analysed directly ex vivo without any additional activating stimulus or artificial sequestering of cytokine by brefeldin A or other Golgi inhibitors. Further analysis of these cells indicated that they were CD11c^{-/low}, CD45RB^{intermediate} and B220⁻ and had plasmacytoid DC morphology. Tolerogenic plasmacytoid DC with similar phenotype but CD45RB^{high} can be generated by in vitro culture with IL-10, can be isolated from the spleens of normal C57B1/6 mice and are elevated in IL10 transgenic mice. The cells take 3 weeks to differentiate in vitro, and in the studies of the present invention appear in CD200KO spleens 3-4 weeks after disease onset suggesting that prolonged stimulation and/or several rounds of cell division are involved. Bone marrow derived plasmacytoid cells were also tolerogenic and capable of generating antigen specific IL-10 secreting Tregs, in vivo. Significant numbers of CD3⁺CD4⁺IL-10⁺ cells were not found in this study, but a trend towards increased numbers of CD3⁺CD4⁺CD25⁺ in CD200KO mice was found and this was significant in tolerised groups at all time points. Tregs can have an immunosuppressive effect, e.g., by inhibiting expression of IL-2 or IL-10. Induction of IL-10 and suppression of IL-2 in all groups at day 28 of the study of the present invention is consistent with induction of regulatory T cells during the disease process, and findings linking nasal administration of antigen with induction of Tr1 (see, e.g., Shevach (2002) Nat. Rev. Immunol 2:389400; McGuirk and Mills (2002) Trends Immunol. 23:450455; Herrath and Harrison (2003) Nat. Rev. Immunol. 3:223-232; Bluestone and Abbas (2003) Nat. Rev. Immunol. 3:253-257; Thornton and Shevach (1998) J. Exp. Med. 188:287-296; Jonuleit, et al. (2000) J. Exp. Med. 192:1213-1222; Wakkach, et al. (2003) Immunity 18:605-617).

Pulmonary DCs mediate immune response to inhaled antigen, inducing T cell hypo-responsiveness to innocuous antigens or preferential activation and expansion of Th2-biased responses. This has been attributed to the mucosal microenvironment and immature phenotype of these cells. IL-10 has a role inducing nasal tolerance and in limiting inflammation later in disease. Th2-derived IL-10 would then have the effect of augmenting tolerance in an antigen specific manner as a single exposure to IL-10 can convert DC to a tolerogenic phenotype (see, e.g., Enk, et al. (1993) J. Immunol. 151:2390-2398; De Smedt, et al. (1997) Eur. J. Immunol. 27:1229-1235; Mitchison, et al. (1999) Springer Semin. Immunopathol. 21:199-210).

III. Purification and Modification of Polypeptides and Nucleic Acids.

The polypeptide and nucleic acid diagnostics and therapeutics of the invention can be prepared by methods established in the art. Purification can involve ion exchange chromatography, immunoprecipitation, epitope tags, affinity chromatography, high pressure liquid chromatography, and use of stabilizing agents, detergents or emulsifiers (Dennison and Lovrien (1997) Protein Expression Purif. 11: 149-161; Murby, et al. (1996) Protein Expression Purif. 7:129-136; Ausubel, et al. (2001) Curr. Protocols Mol. Biol., Vol. 3, John Wiley and Sons, New York, N.Y., pp. 17.0.1-17.23.8; Rajan, et al. (1998) Protein Expression Purif. 13:67-72; Amersham-Pharmacia (2001) Catalogue, Amersham-Pharmacia Biotech, Inc., pp. 543-567, 605-654; Gooding and Regnier (2002) HPLC of Biological Molecules, 2^{nd} ed., Marcel Dekker, NY).

Modifications of proteins, peptides, and nucleic acids, encompass epitope tags, fusion proteins, fluorescent or radioactive groups, monosaccharides or oligosaccharides, sulfate or phosphate groups, C-terminal amides, modified N-terminal amino groups, e.g., by acetylation or fatty acylation, intrachain cleaved peptide bonds, and deamidation products (Johnson, et al. (1989) J. Biol. Chem. 264:14262-14271; Young, et al. (2001) J. Biol. Chem. 276:37161-37165). Glycosylation depends upon the nature of the recombinant host organism employed or physiological state (Jefferis (2001) BioPharm 14:19-27; Mimura, et al. (2001) J. Biol. Chem. 276:45539-45547; Axford (1999) Biochim. Biophys. Acta 1:219-229; Malhotra, et al. (1995) Nature Medicine 1:237-243; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391).

IV. Binding Compositions, Agonists, Antagonists, and Muteins.

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205-6213; He, et al. (1998) J. Immunol. 160:1029-1035; Tang, et al. (1999) J. Biol. Chem. 274:27371-27378; Li, et al. (2002) Immunol. Revs. 190:53-68; Sato, et al. (1994) Mol. Immunol. 31:371-381; Morea, et al. (2000) Methods 20:267-279).

A humanized antibody contains the amino acid sequences from six complementarity determining regions (CDRs) of the parent mouse antibody, which are grafted on a human antibody framework. Alternatives to humanization include use of fully human antibodies, as well as human antibody libraries displayed on phage or human antibody libraries contained in transgenic mice (see, e.g., Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display. A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nat. Biotechnol. 17:397-399).

Humanized antibodies, chimeric antibodies, single chain antibodies, single domain antibodies, bispecific antibodies, and peptide mimetics of antibodies are described (see, e.g., Maynard and Georgiou (2000) Annu. Rev. Biomed. Eng. 2:339-376; Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) New Engl. J. Med. 148:1547-1553; Casset, et al. (2002) Biochem. Biophys. Res. Commun. 307:198-205; U.S. Pat. Nos. 5,932,448; 5,532,210; 6,129,914; 6,133,426; 4,946,778).

Purification of antigen is not necessary for the generation of antibodies. Immunization can be performed by DNA vector immunization, see, e.g., Wang, et al (1997) Virology 228: 278-284. Alternatively, animals can be immunized with cells bearing the antigen of interest followed by hybridoma production, see, e.g., Meyaard, et al. (1997) Immunity 7:283-290; Wright, et al. (2000) Immunity 13:233-242; Preston, et al. (1997) Eur. J. Immunol. 27:1911-1918; Kaithamana, et al. (1999) New Engl. J. Med. 163:5157-5164. Bispecific antibodies are also contemplated (see, e.g., U.S. Pat. Nos. 5,932,448 issued to Tso, et al., 5,532,210 issued to Paulus, and 6,129,914 issued to Weiner, et al.).

Antibody/antigen binding properties can be measured, e.g., by surface plasmon resonance or enzyme linked immunosorbent assay (ELISA) (Neri, et al. (1997) Nat. Biotechnol. 15:1271-1275; Jonsson, et al. (1991) Biotechniques 11:620-627; Hubble (1997) Immunol. Today 18:305-306). The antibodies of this invention can be used for affinity chromatography in isolating the antibody's target antigen and associated bound proteins (Wilchek, et al. (1984) Meth. Enzymol. 104:3-55).

Soluble receptors can be prepared and used according to standard methods (see, e.g., Jones, et al. (2002) Biochim. Biophys. Acta 1592:251-263; Prudhomme, et al. (2001) Expert Opinion Biol. Ther. 1:359-373; Femandez-Botran (1999) Crit. Rev. Clin. Lab Sci. 36:165-224).

Conjugation of antibody, soluble receptor, and other binding compositions to polyethylene glycol (PEG) may result in a prolongation of circulating time and a reduction of antigenicity (Solorzano, et al. (1998) J. Appl. Physiol. 84:1119-1130; Rosenberg, et al. (2001) J. Appl. Physiol. 91:2213-2223; Bendele, et al. (2000) Arthritis Rheum. 43:2648-2659; Trakas and Tzartos (2001) J. Neurochem. 120:4249). Conjugation with PEG may be especially useful for therapeutic antibody fragments, such as Fab′, Fv, F(ab′)₂, and short chain Fv, which tend to have relatively short lifetimes in vivo (Chapman, et al. (1999) Nature Biotechnology 17:780-783).

V. Therapeutic and Diagnostic Uses.

The present invention contemplates the use of agonists or antagonists of CD200 or CD200R to induce or maintain tolerance in various immune or inflammatory disorders. Enhancement or maintenance of tolerance is useful in the treatment of, e.g., graft or transplant rejection; graft versus host disease (GVHD); septic shock; asthma; allergy; and organ specific autoimmune disorders, such as multiple sclerosis, inflammatory bowel disorder (IBD), experimental autoimmune encephalitis (EAE), rheumatoid arthritis, collagen-induced arthritis (CIA), multiple sclerosis, autoimmune myocarditis, nephritis, uveoretinitis, myasthenia gravis, diabetes mellitus, and thryroiditis. IBD includes Crohn's disease, ulcerative colitis, and celiac disease. Tolerance enhancement is also useful in preventing reactions to drugs, e.g., penicillin, recombinant antibodies, and gene therapy to a missing protein; and in promoting maternal tolerance to an embryo or fetus (see, e.g., Whitacre, et al. (1996) Clin. Immunol. Immunopathol. 80: (3 Pt. 2):S31-S39; Murphy and Blazar (1999) Curr. Opin. Immunol. 11:509-515; Efrat (2002) Trends Mol. Med. 8:334-339; Kohm, et al. (2002) J. Immunol. 169:47124716; Thurau and Wildner (2002) Prog. Retin. Eye Res. 21:577-589; Weiner, et al. (1994) Ann. Rev. Immunol. 12:809-837).

Agonists or antagonists of the present invention may be used to treat immune disorders associated with T cells, B cells, mast cells, eosinophils, NK cells, NKT cells, antigen presenting cells (APCs), such as dendritic cells, monocyte/macrophages, endothelial cells, epithelial cells, Peyer's patches or the gut mucosa, or the central nervous system.

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg, see, e.g., Yang, et al. (2003) New Engl. J. Med. 349:427434; Herold, et al. (2002) New Engl. J. Med. 346:1692-1698; Liu, et al. (1999) J. Neurol. Neurosurg. Psych. 67:451456; Portielji, et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg body weight basis.

Formulations of therapeutic and diagnostic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

An effective amount for a particular patient may vary depending on factors such as the condition being treated, the overall health of the patient, the route and dose of administration, and the severity of side affects. Guidance for methods of treatment and diagnosis is available (Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The invention also provides a kit comprising a cell and a compartment, a kit comprising a cell and a reagent, a kit comprising a reagent, a kit comprising a cell and instructions for use or disposal, as well as a kit comprising a cell, compartment, and a reagent. Moreover, the invention also provides a kit comprising a cell and a compartment and instructions, a kit comprising a cell and a reagent and instructions, a kit comprising a and instructions, a kit comprising a cell and instructions, as well as a kit comprising a cell compartment, and a reagent and instructions. The instructions can comprise instructions for use, for disposal of reagents, or for use and disposal.

The broad scope of this invention is best understood with reference to the following examples, which are not intended to limit the inventions to the specific embodiments.

### EXAMPLES

I. General Methods.

Some of the standard methods are described or referenced, e.g., in Maniatis, et al. (1982) Molecular Cloning, A Laboratory Manual, Cold Spring Harbor Press, Cold Spring Harbor, N.Y.; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987) Current Protocols in Molecular Biology and supplements, Greene/Wiley, New York. Methods for protein purification include, e.g., column chromatography, electrophoresis, centrifugation, immunoprecipitation, and cloning and expression by vectors and cells, see, e.g., Amersham Pharmacia Biotech (2003) Catalogue, Piscataway, N.J.; Invitrogen (2003) Catalogue, Carlsbad, Calif.; Sigma-Aldrich (2003) Catalogue, St. Louis, Mo.

Methods for flow cytometry, including fluorescence activated cell sorting (FACS), are available, see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Cell counting can be accomplished with the aid of beads or microspheres, e.g., Caltag® counting beads (Caltag Labs, Burlingame, Calif.) and Perfectcount® (Exalpha Biologicals, Watertown, Mass.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described, see, e.g., Muller-Harmelink (ed.) (1986) Human Thymus. Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology:Text and Atlas, McGraw-Hill, New York, N.Y.

Methods for using animal models, e.g., knockout mice, and cell-based assays for the testing, evaluation, and screening of diagnostic, therapeutic, and pharmaceutical agents are available, see, e.g., Car and Eng (2001) Vet. Pathol. 38:20-30; Kenyon, et al. (2003) Toxicol. Appl. Pharmacol. 186:90-100; Deurloo, et al. (2001) Am. J. Respir. Cell Mol. Biol. 25:751-760; Zuberi, et al. (2000) J. Immunol. 164:2667-2673; Temelkovski, et al. (1998) Thorax 53:849-856; Horrocks, et al. (2003) Curr. Opin. Drug Discov. Devel. 6:570-575; Johnston, et al. (2002) Drug Discov. Today 7:353-363.

Methods for the diagnosis and treatment of inflammatory conditions in animals and in humans are described (Ackerman (1997) Histological Diagnosis of Inflammatory Skin Disease, 2^{nd} ed., Lippincott, Williams, and Wilkins, New York, N.Y.; Gallin, et al. (1999) Inflammation:Basic Principles and Clinical Correlates, 3^{rd} ed., Lippincott, Williams, and Wilkins, New York, N.Y.; Benezra (1999) Ocular Inflammation:Basic and Clinical Concepts, Blackwell Science, Ltd., Oxford, UK; Geppetti and Holzer (1996) Neurogenic Inflammation, CRC Press, Boca Raton, Fla.; Nelson, et al. (2000) Cytokines in Pulmonary Disease:Infection and Inflammation, Marcel Dekker, Inc., New York, N.Y.; O'Byrne (1990) Asthma as an Inflammatory Disease, Marcel Dekker, Inc., New York, N.Y., Parnham, et al. (1991) Drugs in Inflammation (Agents and Actions Suppl. Vol. 32), Springer Verlag, Inc., New York, N.Y.).

Software packages for determining, e.g., antigenic fragments, signal and leader sequences, protein folding, and functional domains, are available, see, e.g., Vector NTI® Suite (Informax, Inc., Bethesda, Md.); GCG Wisconsin Package (Accelrys, Inc., San Diego, Calif.), and DeCypher® (TimeLogic Corp., Crystal Bay, Nev.); Menne, et al. (2000) Bioinformatics 16:741-742. Public sequence databases were also used, e.g., from GenBank and others.

II. Animals and Induction of Experimental Autoimmune Uveoretinitis (EAU).

CD200-deficient mice (CD200^{-/-}) on the C57BL/6 background were generated and isolator reared in a specific pathogen free (SPF) breeding colony established within the biological services Unit of Aberdeen University, UK (Hoek, et al. (2000) Science 290:1768-1771). SPF C57BL/6 wild type (CD200^{+/+}) were purchased from Harlan Olac, UK. Groups of 3-6 mice were used as detailed in the text for each experiment.

For immunization, groups of sex and age matched mice were used at 6-8 weeks of age. Mice were immunized with a s.c. injection of 0.5 mg peptide 1-20 of IRBP (Genosys, Sigma, UK) in Freund's complete adjuvant (FCA, 2.5 mg/ml M. tuberculosis) and given an injection of Bordatella pertussis toxin (PTX) (i.p.) as additional adjuvant (Avichezer, et al. (2000) Invest. Ophthalmol Sci. 41:127-131). At specified times animals were sacrificed by CO₂ asphyxiation, and eyes enucleated for resin histology (haematoxylin and eosin staining for histological scoring system) or for immunocytochemistry. Lymphoid tissue was also sampled to determine peptide specific proliferative and cytokine responses. Severity of disease was assessed using a modified version of the histological grading system for rat EAU. At least three sections from each eye were scored in a masked fashion using a semi-quantitative scoring system that combines the extent of the inflammatory infiltrate and tissue damage in the posterior chamber (Dick, et al. (1994) Eye 8 (pt. 1):52-59).

III. Induction of Nasal Tolerance

Fifty micrograms peptide in 5 microliters PBS or 5 microliters PBS alone was administered intranasally. This regime, administered 10 days prior to immunization is effective in modulating EAU (Jiang, et al. (2001) Br. J. Ophthalmol. 85:739-744). In some experiments mice were sacrificed at intervals over the following 48 hours to examine the effects of intranasal (i.n.) peptide on cells in draining cervical lymph nodes and spleens, or in control submandibular and mesenteric lymph nodes. In other experiments animals were immunised 10 days later with peptide or PBS in FCA with PTX.

IV. Proliferation and Cytokine Profile Assays

Lymphocyte proliferation responses to recall antigen were measured using bromodeoxyuridine (BrDU) flow kit (BD Biosciences, Oxford, UK). This test allows detection of BrDU labeling of cells in the S-phase together with measurement of total DNA content of the cell population as a whole.

Single cell suspensions were obtained from individual spleens by pressing the tissue through a 0.25 mm metal sieve and mononuclear cells purified by Percoll® density gradient centrifugation. Red blood cells were removed by hypotonic lysis. Cultures were set up at a density of 1×10⁶ cells per ml with 10 micrograms/ml peptide 1-20 for 96 hours. This time point was established as optimum by compiling data from 48-120 hour incubations. Each well was then pulsed with 10 microliters of 1 mM bromodeoxyuridine (BrDU) for 45 min, harvested, cells permeabilized with Cytofix/Cytoperm® (BD Biosciences), and frozen at −80° C. in FCS with 10% DMSO prior to staining and analysis. A FITC labeled anti-BrDU antibody was used to identify the extent of BrDU incorporation and the DNA stain 7-amino-actinomycin D (7-AAD) was used to quantify the total DNA content. Measurements were made by 2-color flow-cytometry using a BD FACS Calibur® or BD FACS LSR®. Data was obtained from at least three individual animals at each time point. For cytokine profile analysis of responding cultures, parallel 1 ml cultures were set up with 4×10⁶ cells and 10 micrograms/ml peptide. After 72 hours, supernatants were harvested, clarified by centrifugation, and frozen at −30° until assay. Negative control cultures contained PBS in place of peptide. Concanavalin A at 2.5 micrograms/ml was added to positive control cultures to demonstrate optimal growth conditions and cell viability during the assays.

V. Cytokine Measurements

IL-10 and IL-12 were measured using optELISA® kits from BD Pharmingen, Oxford, UK. The mouse cytokine bead array (CBA kit, BD Pharmingen) was used to measure other Th1/Th2 cytokines. Briefly, the Th1/Th2 CBA assay utilizes five bead populations with distinct fluorescence intensities and coated with capture antibodies specific for murine IL-2, IL-4, IL-5, IFNgamma or TNFalpha. The capture beads were mixed with PE-conjugated detection antibodies and incubated with recombinant standards or test samples to form sandwich complexes. The five bead populations were mixed together and resolved in the FL3 channel of a BD FACsCalibur® flow cytometer.

VI. Serum Antibody Isotype Determination.

Serum was obtained from mice by tail tipping or cardiac puncture and stored frozen at −30° C. until assay. Anti IRBP and peptide 1-20 responses were titrated by ELISA. Ninety-six well plates were coated overnight with 1 micrograms/ml peptide 1-20 in Ca₂CO₃ buffer (pH 9.6). Wells were then washed with 0.5% Tween/PBS and blocked with 1% BSA/PBS. Sera were serially diluted in 1% BSA/0.5% Tween in PBS and incubated for 2 h at 37° C. After washing, bound antibody was detected using peroxidase conjugated rabbit anti-mouse immunoglobulin (Dako, Stockholm, Sweden), O-phenylenediamine (Sigma; 0.4 mg/ml in 0.1 M citrate/acetate buffer pH 6.0), was used as a substrate for the peroxidase reaction. Optical density (OD) was determined at 490 nm on a microtitre plate analyser. Titres were expressed as the reciprocal of the last dilution greater than the pre-immune serum OD.

VII. Immunocytochemistry.

Eyes from immunized mice or lymphoid tissue were dissected, snap frozen in OCT and 7 micrometer serial sections cut, air dried and fixed in 100% cold acetone for immunocytochemistry using the alkaline phosphatase anti-alkaline phosphatase (APAAP) technique. Following re-hydration in TRIS buffered saline (TBS), sections were blocked with TBS 1% normal rabbit serum and then avidin D block solution (Vector Laboratories, Burlingame, Calif.) for polyclonal rabbit anti-STAT 4 (C20; Santa Cruz Biotechnology, CA, USA)) and STAT 6 (M20; Santa Cruz) staining using appropriate controls and blocking peptides as negative controls. Other sections were stained using mouse monoclonal antibodies to CD3 and myeloid cell markers F4/80 antigen (CI:A3-1), MOMA-1 and MOMA-2, from Serotec (Kidlington, Oxford, UK). Activation markers included NOS2 (clone 6; Transduction Laboratories, KY, USA), and CD86 (GL-1), and MHCu class II (I-Ab) (P7.7) both from BD Pharmingen (Coley, Oxford, UK). Positive staining was detected by mouse absorbed biotinylated anti-rabbit Ig-AP or biotinylated anti-mouse Ig-AP conjugate followed by strepavidin:ABC AP complex and fast red substrate (Dako, Stockholm, Sweden) lightly counterstained with haematoxylin. Sections for image analysis were stained in batches to ensure uniform labelling conditions for each antibody. Sections were then analysed using the Aphelion Active X® image analysis program from ADCIS (ADCIS SA, Herouville-Saint-Clair, France). The program was adapted using Visual Basic® (Microsoft, Redmond, Wash.) to allow analysis of immunostaining in user defined regions of the image. An average value (percent of tissue positively stained per×20 field) for each section was obtained from 4-6 fields.

VIII. Flow Cytometry.

A Becton Dickinson (BD) FACS Calibur® was used for data acquisiton and Cell Quest® software (BD) for data analysis. Antigen presenting cells and lymphocytes isolated from lymph nodes and spleens were evaluated by double, or triple immunofluorescence staining with mAbs to the following cell surface markers: CD11b (M1/70), CD11c (HL3), CD4 (RM4-4) CD45RB (16A), CD45R (RA3-6B2), CD40 (3/23), CD86 (GL-1), CD152 (BN13), MHC class II (1-Ab) (AF6-120.1), CD25 (PC61), CD38 (92), CD8a (53-6B2), CD62L (MEL-14) and CD3 epsilon (145-2C11) were all from BD Pharmingen (Coley, Oxford, UK).

For intracellular detection of CTLA-4 and IL-10, quadruple staining was performed. Cells were treated with Cytofix-cytoperm and stained with phycoerythrin (PE) conjugated CD152 (UC10-4F10-11), and PE or FITC conjugated anti-IL-10 (JES5-16E3) following manufacturer's instructions (BD Pharmngen). Anti-CD204 (2F8), F4/80 (CI:A3-1), metallophillic macrophages (MOMA-1) and CD80 (RMMP-1) were from Serotec (Kidlington, Oxford, UK). These were cojugated to FITC, PE, APC, PerCP or biotin as required. Biotin labelled antibodies were detected by addition of SA-APC (1:400) (BD Parmingen). Negative isotype controls, and single positive controls were performed to allow accurate breakthrough compensation.

To examine morphology, cell populations were sorted using a Becton Dickinson FACS DIVA according to gates defined by fluorescent antibody staining for high IL-10, CD11c^{low}, CD11b⁻.

IX. STAT6 Expression and Activation.

Expression and activation of STAT6 was determined by immunoblotting of immunoprecipitates prepared from cytosolic and nuclear extracts (Dick, et al. (2001) Br. J. Ophthalmol. 85:1001-1006). STAT6 was detected using anti-STAT6 antibody (M20, Santa Cruz Biotech., Santa Cruz, Calif.) and Protein A-Sepharose beads to collect STAT6 proteins, followed by separation by SDS-PAGE blotting on a Hybond PVDF membrane (Amersham-Pharmacia) and analysis of blots by probing with anti-STAT6 antibody. Phosphorylated STAT6 was detected using anti-phosphotyrosine antibody (mAb 4G110, Upstate Biotechnol., Charlottesville, Va.).

X. The CD200 Knock Out Enhances Tolerization, as Determined by Histology Score and Splenocyte Proliferation.

Mice were immunized by injection (s.c.) with 0.5 mg IRBP peptide 1-20, where immunization was preceded, by ten days, by intranasal peptide (tolerized group) or control PBS (sham tolerized). Autoimmune uveitis (EAU) was monitored at day 16, day 21, and day 28 post-immunization. Eyes were examined by resin histology or immunochemistry, where the histology score was a composite of two indicia, i.e., inflammatory cell infiltrate and structural tissue damage. Disease onset was accelerated in sham tolerized CD200KO mice, with expression of type 2 nitric oxide synthase (NOS2) in cells of the ciliary body, retinal vascular endothelium, and inner plexiform layer of the retina, with earliest signs of the disease occurring at day 10. But at later times, tissue damage was more severe in the wild type than in the CD200KO mouse (see, Broderick, et al. (2000) Am. J. Pathol. 161:1669-1677).

The tolerization protocol resulted in an improvement in histology score in both the wild type mice and in the CD200KO mice, on days 16, 21, and 28, where this improvement was greater in the CD200KO mice (score 1.7) than in the wild type mice (score 2.9) on day 28. At all time points, tolerization protected both the wild type and CD200KO mouse, where the most highly protected mouse was found at day 28 in the tolerized CD200KO mouse (Table 1).

TABLE 1
Histology score in immunized wild type mice and in immunized
CD200KO mice.

| | | | |
|---|---|---|---|
| | Wild type | | CD200KO |
| Wild type PBS | tolerized | CD200KO PBS | tolerized |

Day 16

| | | | |
|---|---|---|---|
| 1.2 | 0.5 | 2.5 | 1.2 |

Day 21

| | | | |
|---|---|---|---|
| 4.3 | 2.0 | 2.8 | 2.0 |

Day 28

| | | | |
|---|---|---|---|
| 4.8 | 2.9 | 3.3 | 1.7 |

Induction of tolerance in the wild type and CD200KO groups resulted in increases in IgG1 antibody titer, relative to the non-tolerized mice. At day 28, the IgG1 antibody titer was about 6800 in the wild type tolerized mice, and about 7200 in the CD200KO tolerized mice, whereas the IgG1 titer in all non-tolerized mice was about 1800-2000. The tolerance-induced increase in IgG1 indicated a switch from TH1-response to TH2-response.

Proliferation responses by splenocytes to peptide 1-20 showed equivalent responses in the wild type and CD200KO mice (Broderick, et al., supra). In the present tolerization study, splenocyte proliferation was assessed in the four groups of mice, where assessment was by measuring bromodeoxyuridylate labeling of cells in S-phase with measurement of total DNA content of the population as a whole (Table 2). In the present tolerization study, intranasal exposure to antigen had no inhibitory effect on peak proliferation of splenocytes at days 16 or 21, despite reduction in disease in tolerized animals on these two days. By day 28, numbers of cells in S-phase were reduced in both tolerized wild type mice and tolerized CD200KO mice, where this reduction was greater in the CD200KO mice (1.0) than in the wild type mice (9.5) (Table 2). In other words, by day 28 the numbers of cells in S-phase were reduced in both tolerized groups, but more so in the CD200KO tolerized group. Thus, the CD200KO enhances tolerance.

TABLE 2
Percent cells in S phase in immunized wild type mice and in
immunized CD200KO mice.

| | | | |
|---|---|---|---|
| | Wild type | | CD200KO |
| Wild type PBS | tolerized | CD200KO PBS | tolerized |

Day 16

| | | | |
|---|---|---|---|
| 3.5 | 3.0 | 4.0 | 5.0 |

Day 21

| | | | |
|---|---|---|---|
| 9.5 | 22.5 | 11.0 | 12.0 |

Day 28

| | | | |
|---|---|---|---|
| 9.0 | 2.5 | 9.5 | 1.0 |

XI. The CD200 Knock Out Enhances TH2-Type Response but not TH1-Response.

Splenocyte cultures were set up in parallel with the proliferation assays for assessment of cytokine expression, where cytokine expression was measured after re-stimulation with peptide (Tables 3-5). At days 21 and 28, expression of IL-4, IL-5, and IL-10 was greatest in tolerized CD200KO mice, when compared to sham tolerized wild type mice, sham tolerized CD200KO mice, and tolerized wild type mice, demonstrating that CD200KO enhances TH2-response (Tables 3-5).

Interleukin-2 and TNFalpha were elevated in sham tolerized wild type mice at day 16, compared to levels in the other three groups of mice. IL-2 and TNFalpha were also elevated in sham tolerized wild type mice at day 21, compared to levels in the other three groups of mice. At day 28, levels of IL-2 were similar in all four groups, while at day 28 levels of TNFalpha were also similar in all four groups. IL-12 was low in all cultured examined, i.e., about 50 pg/ml or less. Large quantities of IFNgamma (5-15 ng/ml) were also expressed in all cultured examined, but no significant differences between groups were observed.

TABLE 3
IL-4 expression in cultured splenocytes, dependence on
tolerization and CD200KO.

| | | | |
|---|---|---|---|
| Wild type | Wild type | CD200KO | CD200KO |
| PBS | tolerized | PBS | tolerized |

Day 16

| | | | |
|---|---|---|---|
| 35 | 20 | 25 | 80 |

Day 21

| | | | |
|---|---|---|---|
| 80 | 110 | 115 | 240 |

Day 28

| | | | |
|---|---|---|---|
| 30 | 30 | 30 | 50 |

TABLE 4
IL-5 expression in cultured splenocytes,
dependence on tolerization and CD200KO.

| | | | |
|---|---|---|---|
| | Wild type | | CD200KO |
| Wild type PBS | tolerized | CD200KO PBS | tolerized |

Day 16

| | | | |
|---|---|---|---|
| 165 | 20 | 60 | 90 |

Day 21

| | | | |
|---|---|---|---|
| 130 | 60 | 110 | 260 |

Day 28

| | | | |
|---|---|---|---|
| 130 | 60 | 65 | 90 |

TABLE 5
IL-10 expression in cultured splenocytes,
dependence on tolerization and CD200KO.

| | | | |
|---|---|---|---|
| | Wild type | | CD200KO |
| Wild type PBS | tolerized | CD200KO PBS | tolerized |

Day 16

| | | | |
|---|---|---|---|
| 75 | 45 | 40 | 45 |

Day 21

| | | | |
|---|---|---|---|
| 55 | 20 | 30 | 45 |

Day 28

| | | | |
|---|---|---|---|
| 115 | 80 | 100 | 210 |

STAT6 controls the Th2-differentiation pathway. In contrast, STAT4 is activated after IL-12 signaling to drive TH1-type responses (see, e.g., Takeda, et al. (1996) Nature 380:627-630; Kaplan, et al. (1996) Immunity 4:313-319; Kaplan, et al. (1996) Nature 382:174-177; Thierfelder, et al. (1996) Nature 382:171-174). At various time intervals (0, 12, 24, and 48 h) after nasal tolerization, cervical lymph nodes that drain the nasal mucosal lymphoid areas of the spleen (white pulp), and macrophage areas of the spleen (red pulp), were examined for expression and activation of STAT6. Sub-mandibular lymph nodes were analyzed in parallel to serve as a control tissue (Table 6). TH2-type cell-signaling, enhanced by both tolerization and by the CD200 knock out, was demonstrated by increases in STAT6 expression in spleen white pulp and in cervical lymph node, as shown either at t=24 or at 48 h (Table 6). The effect was transient in the cervical nodes but sustained in lymphoid and macrophage areas of the spleen.

STAT4 was transiently increased in draining lymph nodes and spleen of CD200KO mice 24 hours post-treatment. This is consistent with the initial TH1 priming observed in a rat tolerance model, and correlates with increased CD86 expression in cervical lymph nodes. No increase in STAT4 expression was found in the wild type mice, during the sampling period (Table 6) (Dick, et al. (2001) Br. J. Ophthalmol. 85:1001-1006).

TABLE 6
Time course of response to tolerization in immunized
wild type mice and in immunized CD200KO mice.

| | Wild type | | | | CD200 knockout | | | |
|---|---|---|---|---|---|---|---|---|
| Tissue | 0 h | 4 h | 24 h | 48 h | 0 h | 4 h | 24 h | 48 h |

Spleen red pulp (macrophage areas).

| | | | | | | | | |
|---|---|---|---|---|---|---|---|---|
| STAT6 | 19.0 | 42.6 | 46.6 | 52.7 | 25.9 | 36.9 | 48.5 | 56.8 |
| STAT4 | 19.9 | 0.4 | 5.4 | 6.4 | 3.4 | 2.0 | 13.6 | 2.3 |
| CD86 | 17.0 | 32.0 | 15.5 | 18.7 | 15.3 | 21.3 | 13.9 | 26.9 |

Spleen white pulp (lymphoid areas).

| | | | | | | | | |
|---|---|---|---|---|---|---|---|---|
| STAT6 | 3.2 | 5.7 | 23.5 | 13.5 | 3.9 | 4.3 | 12.8 | 32.2 |
| STAT4 | 5.3 | 0.01 | 0.4 | 0.9 | 0.2 | 0.1 | 1.5 | 0.8 |
| CD86 | 5.6 | 3.2 | 2.1 | 6.1 | 8.8 | 3.5 | 4.2 | 11.8 |

Cervical lymph node that drain nasal mucosa.

| | | | | | | | | |
|---|---|---|---|---|---|---|---|---|
| STAT6 | 6.2 | 2.2 | 1.7 | 2.2 | 6.5 | 0.9 | 32.9 | 8.4 |
| STAT4 | 8.0 | 0.7 | 0.3 | 0.3 | 0.4 | 0.3 | 4.0 | 3.4 |
| CD86 | 10.3 | 14.0 | 12.0 | 11.0 | 4.5 | 4.6 | 21.9 | 8.1 |

Submandibular lymph node (control tissue).

| | | | | | | | | |
|---|---|---|---|---|---|---|---|---|
| STAT6 | 9.7 | 7.6 | 1.0 | 1.0 | 4.0 | 18.8 | 8.4 | 12.4 |
| STAT4 | 4.6 | 1.0 | 0.1 | 0.03 | 0.4 | 0.5 | 1.9 | 0.8 |
| CD86 | 8.7 | 6.0 | 3.5 | 10.3 | 1.5 | 5.4 | 1.8 | 6.5 |

Immunostaining of histological sections showed an increase in STAT6 expression in the CD200KO mice, demonstrating the phenomenon of CD200KO-dependent expression of STAT6 (Table 7). Related studies of cervical lymph nodes demonstrated that STAT6 was translocated from the cytosol to the nucleus in the CD200KO mice, but not in the wild type mice, and that STAT6 phosphorylation was greater in the CD200KO mice than in the wild type mice.

TABLE 7
Percentage of histological section of tissue expressing STAT6, at 24 h
or 48 h after tolerization.

| | Wild type mice | CD200 knockout mice |
|---|---|---|
| Cervical lymph note (24 h) | 1.7 | 32.9 |
| Spleen (48 h) | 13.5 | 32.2 |

Small increases in STAT6 were found with tolerization in the submandibular lymph node of CD200KO mice. These small increases may have been produced by ingestion of small amounts of antigen during intranasal administration, to systemic effects of the changes in spleen, or to effects of disseminated antigen (Dick, et al., supra).

XII. CD200KO Induces Increases in Tregs and Increases in IL-10 Positive Cells of Respiratory Tract Dendritic Cells.

Respiratory tract dendritic cells (DCs) are implicated in tolerance to nasally-administered antigens, and preferentially stimulated TH2-type response (Akbari, et al. (2001) Nat. Immunol. 2:725-731; Stumbles, et al. (1998) J. Exp. Med. 188:2019-2031). CD45⁺ cells from the respiratory tracts of wild type and CD200KO mice were isolated. In wild type mice, the major population was CD11c⁺ DCs (over 80%), with few CD11b⁺ cells (10-15%). In CD200KO mice, the major population was CD11b⁺ cells (over 40%), with fewer CD11c⁺DCs (35-40%). The cells from both wild type and CD200KO mice showed low levels of activation markers, as expected for respiratory tract APCs. Both CD11b⁺ and CD11c⁺ cells from CD200KO respiratory tract expressed lower levels of MHC class II antigen than the corresponding cells from wild type mice. F4/80 was found on about 25-28% of the CD45⁺ cells in both wild type and CD200KO mice, while CD204 was found on about 15% of CD45⁺ cells from wild type mice, and about 5% of CD45⁺ cells from CD200KO mice.

TH2-type response can provoke in increase in regulatory T cells (Tregs), while tolerization can be dependent on IL-10 and on Tregs (see, e.g., Kaya, et al. (2002) J. Immunol. 168:1552-1556; Massey, et al. (2002) Vet. Immunol. Immunopathol. 87:357-372; Akbari, et al. (2001) Nat. Immunol. 2:725-731; Kohm, et al. (2002) J. Immunol. 169:4712-4716). Fluorescence measurements on non-permeabilized spleen cells of control mice and CD200KO mice demonstrated moderate increases in CD4⁺CD25⁺ Tregs, within the CD3⁺ T cell population, where these increases were provoked by the CD200KO knockout (Table 8). At all three time points, the tolerized, CD200KO mice had a greater percentage of CD4⁺CD25⁺ Tregs than the tolerized, wild type mice, demonstrating the dependence on CD200 for regulatory T cell response (Table 8).

The percentage of CD3⁺ cells that are CD4⁺CD25⁺ cells in normal, wild type mice (not immunized; not tolerized) was about 8.7%, and in CD200KO mice (not immunized; not tolerized) was about 9.2% (data not shown).

TABLE 8
Influence of tolerization and the CD200KO on the percentage of
CD3⁺cells that are CD4⁺CD25⁺ cells, with analysis of splenocytes.

| | | | |
|---|---|---|---|
| | Wild type | | CD200KO |
| Wild type PBS | tolerized | CD200KO PBS | tolerized |

Day 16

| | | | |
|---|---|---|---|
| 10.5 | 9.8 | 11.0 | 13.2 |

Day 21

| | | | |
|---|---|---|---|
| 10.1 | 9.8 | 12.2 | 12.9 |

Day 28

| | | | |
|---|---|---|---|
| 13.0 | 11.8 | 13.9 | 15.0 |

Interleukin-10 producing cells were found in the spleen myeloid cell population. Two main populations were found from day 21 onwards, i.e., IL-10^{low}CD11b⁺ and IL-10^{high}CD11b⁻ cells. The percentage of IL-10^{low}CD11b⁺ cells ranged from 2-8%, with a trend to higher percentages at day 28, while the IL-10^{high}CD11b⁺ cell population was smaller, about 2% at day 21 and 4% at day 28. Analysis by the geometric fluorescence index demonstrated that cells from both sham tolerized CD200KO mice and tolerized CD200KO mice produced greater levels of IL-10 than wild type sham tolerized and wild type tolerized mice. Analysis of the IL-10^{high}CD11b⁺ cells at day 28 demonstrated that most of these cells were CD11c^{-/low}, CD3⁻, B220⁻, and CD45Rb^{intermediate}, and showed a classic plasmacytoid DC morphology. This phenotype is similar to a CD11c^{low}CD45RB^{high} subset of plasmacytoid DCs, generated in vitro, that can induce tolerance and differentiation of Tr1 cells in vivo (Wakkach, et al. (2003) Immunity 18:605-617).

XIII. Expression of CD200R as Determined by Real Time PCR.

CD200R expression was determined by real time PCR analysis by Taqman® assays (PE Applied Biosystems, Foster City, Calif.), where results are relative to ubiquitin expression (Table 9). The increases in CD200R expression found in TH2 cells indicates that these cells can be modulated by treatment with an agonist or antagonist of the CD200/CD200R signaling pathway. Antagonists of the CD200/CD200R pathway, such as an anti-CD200 antibody or a CD200R knockout, provoke increases in TH2 response.

TABLE 9
Expression of CD200R by Taqman ® analysis; relative to
ubiquitin = 1.0.

| | |
|---|---|
| Mouse BALB/c T cell TH1 activated IFNg/IL-12/anti-IL-4 pool | 22 |
| pool Mel 14+ polarized. | |
| Mouse BALB/c T cell TH2 activated IL-4/anti-IFNg pool | 62 |
| Mel 14+ polarized. | |
| Mouse BALB/c T cell TH1 activated aCD3 pool Mel14 br CD4+ | 50 |
| 1 week polarized. | |
| Mouse BALB/c T cell TH2 activated aCD3 pool Mel14 br CD4+ | 387 |
| 1 week polarized. | |
| Mouse BALB/c T cell TH1 fresh 3 week polarized. | 37 |
| Mouse BALB/c T cell TH2 fresh 3 week polarized. | 392 |
| Mouse BALB/c T cell TH1 activated PMA/ionomycin 3X | 23 |
| polarized cells. | |
| Mouse BALB/c T cell TH2 activated PMA/ionomycin 3X | 301 |
| polarized cells. | |
| Mouse C57BL/6 T cell TH1 activated PMA/ionomycin 3 | 23 |
| week polarized. | |
| Mouse C57BL/6 T cell TH2 activated PMA/ionomycin 3 | 735 |
| week polarized cells. | |

Many modifications and variations of this invention, as will be apparent to one of ordinary skill in the art can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

What is claimed is:

1. A method of modulating tolerance to an antigen in a subject with an inflammatory or immune condition or disorder, comprising treating with an agonist or antagonist of CD200.

2. The method of claim 1, wherein the modulating increases or maintains tolerance and the treatment:
a) comprises administering an antagonist of CD200; or
b) increases TH2-type response.

3. The method of claim 1, wherein the agonist or antagonist is derived from the antigen binding site of an antibody that specifically binds to CD200 or to CD200R.

4. The method of claim 3, wherein the antagonist is an antibody that specifically binds to:
a) CD200; or
b) CD200R.

5. The method of claim 3, wherein the agonist or antagonist comprises:
a) a polyclonal antibody;
b) a monoclonal antibody;
c) a humanized antibody;
d) an Fv, Fab, or F(ab′)₂ fragment; or
e) a peptide mimetic of an antibody.

6. The method of claim 1, wherein the agonist or antagonist comprises a nucleic acid that:
a) encodes a CD200 or CD200R; or
b) specifically binds a polynucleotide encoding a CD200 or CD200R.

7. The method of claim 6, wherein the nucleic acid comprises:
a) an anti-sense nucleic acid;
b) an RNA interference nucleic acid; or
c) a genetic mutation in the genome of the subject that reduces expression of biologically active CD200 or CD200R.

8. The method of claim 1, wherein the condition or disorder comprises an autoimmune condition or disorder.

9. The method of claim 1, wherein the condition or disorder comprises:
a) uveoretinitis;
b) graft or transplant rejection;
c) diabetes mellitus;
d) multiple sclerosis;
e) inflammatory bowel disorder (IBD);
f) rheumatoid arthritis; or
g) asthma or allergy.

10. The method of claim 1, wherein tolerance is induced:
a) intranasally;
b) enterally;
c) orally;
d) parenterally;
e) intravenously; or
f) mucosally.

11. The method of claim 2, wherein the increase or maintenance comprises an improvement in a histological score.

12. The method of claim 11, wherein the improvement comprises a reduction in:
a) inflammatory cell infiltrate; or
b) structural tissue damage.

13. The method of claim 12, wherein:
a) the cell infiltrate is in a retina; or
b) the tissue damage is of a photoreceptor cell.

14. The method of claim 11, wherein the disorder or condition results from an immunization.

15. The method of claim 2, wherein the TH2-type response comprises a detectable increase in expression or levels of a cytokine that is:
a) IL-4;
b) IL-5;
c) IL-10; or
d) IL-13.

16. The method of claim 15, wherein expression or levels of the TH2 cytokine is at least 2-fold greater with CD200 antagonist treatment than without CD200 antagonist treatment.

17. The method of claim 1, wherein immune cell proliferation is detectably decreased or inhibited in a tolerized subject treated with a CD200 antagonist, relative to a tolerized subject not treated with a CD200 antagonist.

18. The method of claim 17, wherein immune cell proliferation with CD200 antagonist treatment is:
a) 75% or less; or
b) 50% or less,
than proliferation without CD200 antagonist treatment.

19. The method of claim 17, wherein the immune cell is a splenocyte.

20. The method of claim 1, wherein the CD200 antagonist treatment results in a detectable increase in expression or activation of STAT6 with treatment with the CD200 antagonist, as compared with treatment without the CD200 antagonist.

21. The method of claim 1, wherein there is a detectable increase in activity or levels of:
a) T regulatory cells (Tregs); or
b) IL-10-expressing cells;
with treatment with the CD200 antagonist, as compared with treatment without the CD200 antagonist.

22. The method of claim 21, wherein the:
a) Tregs comprise CD3⁺CD4⁺CD25⁺ T cells; or
b) the IL-10 expressing cells are:
i) CD11b⁻;
ii) CD11b⁻, CD11c^{-/low}, CD3⁻, B220⁻, CD45RB^{intermediate}; or
iii) plasmacytoid dendritic cells.

23. The method of claim 1, wherein the modulating is decreasing and the treating comprises an agonist of CD200.

24. The method of claim 23, wherein the immune condition or disorder is:
a) persistent infection;
b) or cancer.

25. The method of claim 23, wherein the modulation:
a) decreases TH2 response; or
b) decreases or inhibits activity or levels of regulatory T cells (Tregs).
